(19) United States
(12) Patent Application Publication
(10) Pub. No.: US 2026/0335515 A1
(43) Pub. Date: Oct. 29, 2026

(54) POINT OF USE DEVICE COATING

(71) Applicant: PMIDG, LLC, Anderson, SC (US)

(72) Inventors: David M. Gravett, Palm Coast, FL (US); Kara Bethany Acampora, West Union, SC (US)

(21) Appl. No.: 19/491,145

(22) PCT Filed: Jun. 7, 2024
(86) PCT No.: PCT/US2024/033019

§ 371 Date: Dec. 8, 2025

Related U.S. Application Data
(60) Provisional application No. 63/471,570, filed on Jun. 7, 2023.

(51) Int. Cl. A61L 31/10; A61L 31/14

(52) U.S. Cl. CPC A61L 31/10; A61L 31/14; A61L 2400/10

(57) ABSTRACT

The present disclosure relates compositions, kits, and methods of use thereof for preparing medical devices that can be coated with an aqueous lubricious compositions that can be used to coat implantable medical devices at the point of use to allow easy insertion of the medical device into the surgically created site.

### CROSS-REFERENCE TO RELATED APPLICATION

This PCT application claims priority to, and the benefit of, U.S. Provisional Patent Application No. 63/471,570, filed Jun. 7, 2023, entitled “POINT OF USE DEVICE COATING,” which is incorporated by reference herein in its entirety.

### FIELD

The present disclosure relates compositions, kits, and methods of use thereof for preparing medical devices that can be coated with a biocompatible, degradable lubricant.

### BACKGROUND

Many medical devices are required to be inserted into the body. Often the insertion of these devices is hindered by the friction of the device surface against the tissue or opening through which the device is being introduced. This can lead to trauma to the tissue as well as the potential to entrain surface associated entities into the insertion site. Compositions of polymers exist to assist in the passage of medical devices through an insertion site. These polymers are biocompatible but tend to be non-degradable. As such these compositions cannot be used to coat medical devices that are inserted through an insertion site such as a surgical incision or trauma site.

What is needed is a means to facilitate the easy insertion of the medical device to the desired location.

### SUMMARY

In brief, the present disclosure provides compositions, kits, methods of preparing compositions, and methods of using the compositions and kits disclosed herein.

The above-mentioned additional features of the present disclosure and the manner of obtaining them will become apparent, and the disclosure will be best understood by reference to the following more detailed description. All references disclosed herein are hereby incorporated by reference in their entirety as if each was incorporated individually.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The present disclosure provides compositions, kits, and methods of coating a medical device with a biocompatible, degradable lubricant to facilitate the easy insertion of the medical device to a desired location.

In some aspects, disclosed herein is a kit comprising a sterile aqueous lubricant and a sterile medical device, wherein the sterile aqueous lubricant comprises a biocompatible, degradable polymer comprising hyaluronic acid, sodium hyaluronate, octanethiol derivatized hyaluronic acid, mercaprobenzoic acid derivatized hyaluronic acid, a hyaluronic acid derivative, or a hyaluronate derivative thereof.

In some embodiments, the kit further comprises a sterile container into which the sterile aqueous lubricant can be dispensed. In some embodiments, the sterile medical device comprises a biologically active agent, including but not limited to an antibiotic, an antiviral, a polypeptide, a polynucleotide, a hormone, an antibody-drug conjugate, an anti-cancer agent, a small molecule inhibitor, and any therapeutic agent thereof.

In some embodiments, the sterile medical device comprises a mesh, a catheter, a pouch, a cardiac implantable electronic device, a needle, a syringe, a stent, a suture, a replacement tissue, or a composition or device thereof.

In some embodiments, the sterile medical device further comprises a degradable copolymer selected from one or more monomers consisting of lactide, glycolide, ε-caprolactone, trimethylene carbonate, morpholine-dione, p-dioxanone and 1,5-dioxapan-2-one.

In some aspects, disclosed herein is a composition comprising the sterile aqueous lubricant of any preceding aspect.

In some embodiments, the composition further comprises a sterile carrier selected from water, saline, buffer, solubilizer, stabilizer, or a combination thereof.

In some aspects, disclosed herein is a method of placing a coated sterile medical device into a subject, the method comprising applying a sterile aqueous lubricant to a sterile medical device, wherein the sterile aqueous lubricant comprises a biocompatible, degradable polymer and a sterile carrier, and wherein the polymer comprises hyaluronic acid, sodium hyaluronate, octanethiol derivatized hyaluronic acid, mercaprobenzoic acid derivatized hyaluronic acid, a hyaluronic acid derivative, or a hyaluronate derivative thereof, inserting the coated sterile medical device into a surgically created site in the subject, and closing the surgically created site that now contains the coated sterile medical device.

In some aspects, disclosed herein is a method of placing a coated sterile medical device into a subject, the method comprising applying a sterile aqueous lubricant to a sterile medical device, wherein the sterile aqueous lubricant comprises a biocompatible, degradable polymer and a sterile carrier, and wherein the polymer comprises hyaluronic acid, sodium hyaluronate, octanethiol derivatized hyaluronic acid, mercaprobenzoic acid derivatized hyaluronic acid, a hyaluronic acid derivative, or a hyaluronate derivative thereof, inserting the coated sterile medical device into a surgically created site in the subject, irrigating the surgically created site that contains the coated medical device with saline, and closing the surgically created site that now contains the coated sterile medical device.

In some embodiments, the method of any preceding aspect comprises the sterile carrier being selected from water, saline, buffer, solubilizer, stabilizer, or a combination thereof. In some embodiments, the method of any preceding aspect further comprises a sterile container into which the sterile aqueous lubricious composition can be dispensed.

In some embodiments, the method of any preceding aspect comprises the coated sterile medical device comprising a biologically active agent, including but not limited to an antibiotic, an antiviral, a polypeptide, a polynucleotide, a hormone, an antibody-drug conjugate, an anti-cancer agent, a small molecule inhibitor, or any therapeutic agent thereof.

In some embodiments, the method of any preceding aspect comprises the sterile medical device comprising a mesh, a catheter, a pouch, a cardiac implantable electronic device, a needle, a syringe, a stent, a suture, a replacement tissue, or a composition or device thereof.

In some embodiments, the method of any preceding aspect comprises the sterile medical device further comprising a degradable copolymer selected from one or more monomers consisting of lactide, glycolide, ε-caprolactone, trimethylene carbonate, morpholine-dione, p-dioxanone and 1,5-dioxapan-2-one.

The details of one or more of the preceding aspects are set forth in the description below. The features illustrated or described in connection with one exemplary aspect may be combined with the features of other aspects. Thus, any of the various aspects described herein can be combined to provide further aspects. Aspects of the aspects can be modified, if necessary to employ concepts of the various patents, applications and publications as identified herein to provide yet further aspects. Other features, objects and advantages will be apparent from the description, the drawings, and the claims.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

The present disclosure may be understood more readily by reference to the following detailed description of preferred aspects of the disclosure and the Examples included herein. In an aspect, the present disclosure provides an aqueous lubricious composition for coating medical devices. The coating process may occur at the point of care and the coated device is used immediately without any significant drying of the applied coating.

Polymers that can be used to prepare a disclosed aqueous lubricious composition including but are not limited to synthetic polymers and natural polymers. These polymers can be non-degradable or degradable, also referred to herein as absorbable or unabsorbable, or resorbable or non-resorbable, intending that the material will be degraded by physiological conditions. In one aspect, a polymer can be a polysaccharide. Exemplary polysaccharide polymers useful in the present disclosure include, but are not limited to, sodium alginate, calcium alginate, derivatized alginate, dextran, derivatized dextran, carboxymethyl cellulose, hydroxypropyl cellulose, hydroxypropyl methylcellulose, hyaluronic acid, hyaluronic acid derivatives, heparin, chitosan, derivatized chitosan, xantham gum, Xylan, guar gum, pullulan or locust bean gum. Other exemplary polymers useful in the present disclosure include, but are not limited to, poly acrylic acid polymers and copolymers, poly(hydroxyethyl methacrylate) polymers and copolymers, polyethylene glycol polymers and copolymers.

Polyethylene glycol polymers may include but are not limited to linear polymers, branched polymers, brush-like polymers and multi-armed polymers and block copolymers. Polyethylene glycol multi-arm polymers can include polymers with 3-arms, 4-arms, 5-arms, 6-arms and 8-arms. Polyethylene glycol copolymer can include but are not limited to polymers that contain one or more residues of amino acids, one or more residues of cyclic monomers such as 1-lactide, dl-lactide, glycolide, trimethylene carbonate, epsilon-caprolactone, p-dioxanone and a morpholinedione. In one aspect, the polyethylene glycol copolymers can be blocking copolymers. In another aspect the polyethylene glycol block copolymers can comprise blocks of polyproylene glycol.

Hyaluronic acid derivatives can include but are not limited to ester derivatives, amide derivatives, urethane derivatives, ether derivatives. Hyaluronic ether derivatives can include but are not limited to derivatives described in U.S. Pat. No. 11,440,976 which is incorporated herein by reference in its entirety for its teachings. In an aspect, hyaluronic acid derivatives comprise one or more alkyl groups that are chemically bound to a hydroxyl group of the hyaluronic acid through an ether bond. In another aspect, the hyaluronic acid derivatives comprise one or more alkyl groups that are chemically bound to a hydroxyl group of the hyaluronic acid through the residue of a divinyl sulfone linker. Examples of these derivatives are described in U.S. Pat. No. 11,440,976 which is incorporated herein by reference in its entirety for its teachings. “Alkyl” refers to a straight or branched hydrocarbon chain radical consisting solely of carbon and hydrogen atoms, containing no unsaturation, having from one to the specified number of carbon atoms, and which is attached to the rest of the molecule by a single bond, e.g., methyl, ethyl, n propyl, 1 methylethyl (iso propyl), n butyl, n pentyl, 1,1 dimethylethyl (t butyl), 3 methylhexyl, 2 methylhexyl, and the like. In one aspect, the hyaluronic acid derivatives may comprise one or more aromatic groups that are chemically bound to a hydroxyl group of the hyaluronic acid through an ether bond. In an aspect, hyaluronic acid derivatives may comprise one or more aromatic groups that are chemically bound to a hydroxyl group of the hyaluronic acid through the residue of a divinyl sulfone linker. An aromatic moiety refers to a carbocyclic aromatic moiety, a.k.a., an aryl moiety, or a heteroaromatic moiety, a.k.a., a heteroaryl moiety, either having 1-20 carbon atoms, the heteroaromatic moiety having at least one heteroatom selected from sulfur, oxygen and nitrogen. Examples of these derivatives are described in U.S. Pat. No. 11,440,976 which is incorporated herein by reference in its entirety for its teachings.

In an aspect, hyaluronic acid derivatives that that can be used for disclosed aqueous lubricious composition are hyaluronic acid/polyanionic polysaccharides combinations as described in U.S. Pat. No. 5,017,229 which is incorporated herein by reference in its entirety for its teachings.

In an aspect, a disclosed aqueous lubricious composition may comprise more than one polymer. In an aspect, a disclosed aqueous lubricious composition may comprise polymers that have different molecular weights. In an aspect, a disclosed aqueous lubricious composition may comprise polymers that have a similar chemical structure but have different molecular weights. In an aspect, the polymers used to prepare the coating solution are biocompatible and degradable such that if any residual polymer remains at the site if insertion, this will not result in any biocompatibility or toxicity to the surrounding tissue of distant organs.

In some embodiments, the hyaluronic acid derivative can be a hyaluronic acid functionalized at the primary hydroxyl of the N-acetyl-D-glucose residue, the carboxylic acid of the D-gluconic acid residue, or a combination thereof. The degree of derivatization may be expressed as a molar ratio between functionalized N-acetyl-D-glucose residues and unfunctionalized N-acetyl-D-glucose residues. The ratio may be determined using conventional techniques such as NMR. In some embodiments, the degree of derivatization may be expressed as a molar ratio between the amount of derivatizing agent and hyaluronic acid starting residues, under the assumption that the derivatization reaction proceeds to 100% completion. In certain embodiments, the hyaluronic acid derivative can have a degree of derivatization that is from 1-99%, from 1-50%, from 1-25%, from 1-10%, from 1-5%, from 0.1-2.5%, from 1-2.5%, from 2.5-5%, from 2.5-7.5%, from 5-7.5%, from 5-10%, from 5-15%, from 7.5-12.5%, from 10-15%, from 10-20%, from 10-25%, from 10-50%, from 25-50%, or from 50-99%.

The hyaluronic acid derivative can have a molecular weight that is from 100-3,000 kDa, from 100-2,000 kDa, from 100-1,000 kDa, from 100-500 kDa, from 100-250 kDa, from 250-500 kDa, from 250-750 kDa, from 500-1,000 kDa, from 500-1,500 kDa, from 750-1,250 kDa, from 1,000-1,500 kDa, or from 1,000-2,000 kDa. For embodiments in which the hyaluronic acid derivative is a crosslinked hyaluronic acid derivative, the molecular weight refers to the mass prior to the crosslinking reaction.

In some embodiments, the hyaluronic acid derivative can include residues of Formula (1R):

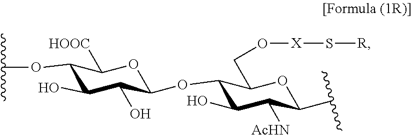

wherein X has the formula:

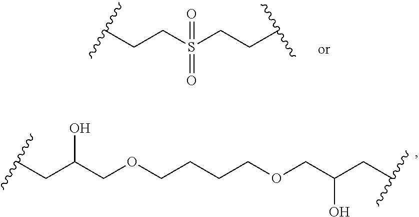 [SMILES: *CCS(=O)(=O)CC* ; *CC(O)COCCCCOCC(O)C*]

and
R is a C₁₋₂₀alkyl, C₃₋₂₀cyclolkyl, C₆₋₁₂aryl, C₁₋₂₀heterocyclyl, or C₁₋₂₀heteroaryl, wherein R is optionally substituted one or more times by halo, OH, COOH, NH₂, OC₁₋₃alkyl, [OCH₂CH₂]ₙOR^{p}, wherein n is 1-200, 1-100, 1-50, 1-25, 1-10, 5-15, 10-25, 10-50, 25-50, 25-75, 50-100, or 100-200, and R^{p} is H or CH₃. In certain implementations R is a C₁₋₁₂alkyl, a C₈₋₁₂alkyl, a C₈₋₁₆alkyl, a C₁₂₋₁₆alkyl, a C₄₋₈alkyl, or a C₅₋₈cycloalkyl. In certain implementations R is a linear C₁₋₁₂alkyl, a C₈₋₁₂alkyl, a C₈₋₁₆alkyl, a C₁₂₋₁₆alkyl, a C₄₋₈alkyl, or a C₅₋₈cycloalkyl. In some embodiments, R is n-pentyl, n-hexyl, n-heptyl, n-octyl, n-nonyl, n-decyl, n-undecyl, or n-dodecyl. In some embodiments, R is methyl, ethyl, n-propyl, isopropyl, n-butyl, isobutyl, sec-butyl, or t-butyl. In certain embodiments, R is substituted one time by COOH. In certain embodiments, R is phenyl, optionally substituted one or more times by COOH, OH, or NH₂. In some embodiments, R is 2-carboxyphenyl, 3-carboxyphenyl, or 4-carboxyphenyl.

In certain embodiments, the hyaluronic acid derivative can include residues of Formula (1U):

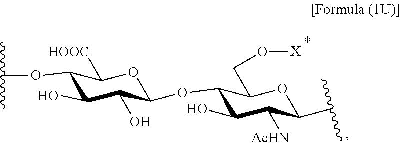

Wherein X* has the formula:

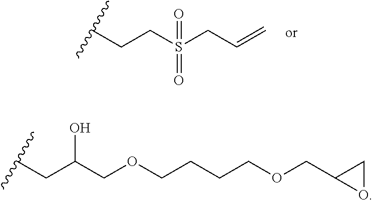

In certain embodiments, the molar ratio of residues of Formula (1R) to residues of Formula (1U) can be 250:1 to 1:1, 250:1 to 50:1, 250:1 to 100:1, 100:1 to 10:1, 100:1 to 50:1, 50:1 to 25:1, 25:1 to 1:1, 25:1 to 10:1, 10:1 to 5:1 or 5:1 to 1:1.

The hyaluronic acid derivative may be obtained by reacting hyaluronic acid under conditions suitable to form residues of Formula (1U) (e.g., with divinyl sulfone and/or BDDE), and then reacting those residues with HS-R, wherein R is as defined above. The hyaluronic acid may also be partially crosslinked using these reagents as well. Exemplary processes are described in U.S. Pat. No. 11,440,976

In some embodiments, the hyaluronic acid derivative can be a crosslinked hyaluronic acid derivative. In other embodiments, the hyaluronic acid derivative is not substantially crosslinked. In certain embodiments, the hyaluronic acid derivative is not substantially crosslinked and has a viscosity that is substantially the same as the initial hyaluronic acid used to prepare the hyaluronic acid derivative.

Medical devices that can be coated with a disclosed composition herein include but are not limited to a catheter, a needle, a biopsy needle, a suture, a trocar, a tissue marker, a guide wire, and endoluminal sheath, a suture, a braid, a trocar, a mesh pouch, a hernia mesh, a surgical mesh, a mesh for breast reconstruction, a contact lens, an intra-ocular lens, a stent (for example vascular stent, esophageal stent, biliary stent coronary stent, renal stent, peripheral vascular stent, urinary stent, ureteral stent), a nasal splint, a vascular graft, a stent-graft, aneurysm coils, introducer sheaths, balloon catheters, vascular closure devices, inferior vena cava filter, an artificial joint, a replacement tendon, a tendon repair patch, a device that is used to achieve spinal fusion device, a pace maker, pace maker leads, cardiac implantable electronic devices, implantable cardioverter defibrillators, spinal cord stimulator, leads for implantable electronic devices, and hydrocephalic shunts. In an aspect, a medical device can be a pouch or envelope into which another medical device is inserted. In an aspect, the pouch or envelope comprises a degradable polymer. In an aspect, degradable polymers are synthesized from one or more monomers of l-lactide, dl-lactide, glycolide, ε-caprolactone, trimethylene carbonate, morpholine-dione, p-dioxanone and 1,5-dioxapan-2-one. In an aspect, a degradable polymer comprises a glycolide copolymer. In an aspect, the degradable polymer comprises a lactide copolymer. In an aspect, the degradable polymer comprises a polydioxanone polymer or copolymer. In an aspect, the degradable polymer tyrosine based polymer. Examples of tyrosine based polymers are described in US U.S. Pat. No. 6,120,491 which is incorporated herein by reference in its entirety for its teachings. In an aspect, a degradable polymer polyacrylate polymer comprises a tyrosine residue. In an aspect, a degradable polymer is a polyhydroxyalkanoate. A polyhydroxyalkanoate polymer may include but is not limited to poly 3-hydroxybutyrate, poly-4-hydroxybutyrate, polyhydroxyvalerate, polyhydroxyoctanoate, polyhydroxyhexanoate and copolymers thereof.

In an aspect, a medical device pouch or envelope comprises a non-degradable polymer. In an aspect a non-degradable polymer is polypropylene, polyethylene, polytetrafluoroethylene, polyethylene terephthalate or a combination thereof. In an aspect, a pouch or envelope comprises an acellular biological matrix. In an aspect, a pouch or envelope comprises porcine small intestine submucosa extracellular matrix. In an aspect, a medical device and/or a medical device pouch or envelope may comprise two or more different polymers. In an aspect, different polymers can be degradable, non-degradable or a combination thereof. In an aspect, the medical device comprises a metal. In another aspect, a medical device and/or a medical device pouch or envelope may comprise a metal and a polymer. In an aspect, a medical device and/or a medical device pouch or envelope may comprise a combination of more than one medical device. For example, a medical device may comprise a cardiac implantable electronic device that is inserted into a pouch or envelope. In an aspect, a medical device and/or a medical device pouch or envelope is sterile. In an aspect, a medical device and/or a medical device pouch or envelope may further comprise a biologically active agent. In an aspect, a medical device and/or a medical device pouch or envelope can further comprise more than one biologically active agents.

In another aspect of the invention, a medical device and/or a medical device pouch or envelope is sterile and inside a container that maintains sterility for at least 6 months. In an aspect, the container is but is not limited to a foil pouch, a Tyvek pouch, a thermoformed container, or a thermoformed container sealed with a Tyvek layer. In an aspect, the sterile medical device and/or a medical device pouch or envelope is within the sealed container.

A kit of the present disclosure can comprise a sterile medical device and a polymer that can be used to prepare the aqueous lubricious composition. In an aspect, a kit of the present disclosure can comprise a sterile medical device, and a sterile polymer that can be used to prepare a disclosed aqueous lubricious composition. In an aspect, a kit of the present disclosure may comprise a sterile medical device, a polymer that can be used to prepare a disclosed aqueous lubricious composition and an aqueous solution that will solubilize the polymer to form a disclosed aqueous lubricious composition. In an aspect, a kit of the present disclosure can comprise a sterile medical device, a sterile polymer that can be used to prepare a disclosed aqueous lubricious composition and a sterile aqueous solution that will solubilize the polymer to form a disclosed aqueous lubricious composition. In an aspect, a kit of the present disclosure can comprise a sterile medical device and a sterile aqueous lubricious composition.

A disclosed aqueous lubricious composition may comprise a polymer and an aqueous solution. In an aspect, a disclosed aqueous lubricious composition may further comprise one or more of a buffer, complexing agent, tonicity modulator, ionic strength modifier, solvent, anti-oxidant, preservative, viscosity modifier, pH modifier, surfactant, emulsifier, phospholipid, stabilizer or a combination thereof.

Buffers that can be used include aqueous solutions prepared using one or more of the following materials: potassium hydrogen phthalate, sodium hydrogen phthalate, potassium or sodium dihydrogen phosphate, dipotassium or disodium hydrogen phosphate, phosphoric acid, boric acid, sodium acetate, acetic acid, ammonium chloride, ammonium acetate, (4-(2-hydroxyethyl)-1-piperazineethanesulfonic acid).

Complexing agents can include but are not limited to _α-cyclodextrin, _β-cyclodextrin (2-Hydroxypropyl)-Beta-Cyclodextrin, Sulfobutylether Beta Cyclodextrin Sodium, Ethylenediaminetetraacetic acid (EDTA)

Phospholipids that can be used include but are not limited to hydrogenated soy phosphatidylcholine, distearoylphosphatidylglycerol, L-α-dimyristoylphosphatidylcholine, L-α-dimyristoylphosphatidylglycerol

Surfactants that can be used include ionic and non-ionic surfactants. Ionic surfactants can include cationic, anionic and zwitterionic surfactants. Non-ionic surfactants can include but are not limited to (Cremophor EL, Cremophor RH 40, Cremophor RH 60, d-_-tocopherol polyethylene glycol 1000 succinate, Brij, Myrj, polysorbate 20, polysorbate 80, polysorbate 40, polysorbate 60, polysorbate 65, polysorbate 85, Solutol HS 15, sorbitan monooleate (Span 80), Sorbitan monopalmitate (Span 40), sorbitan monostearate (Span 60), sorbitan trioleate (Span 8) poloxamer 407, Labrafil M-1944CS, Labrafil M-2125CS, Labrasol, Gellucire 44/14, nonoxynol-9, Softigen 767, octyl beta-D-glycopyranoside (OGP), hexyl beta-D-glucopyranoside (HGP), Octyl beta-D-1-thioglucopyranoside (TGP), Decyl-beta-D-glucopyranoside (DGP), Dodecyl-beta-D-glucopyranoside (DdGP), N-octyl beta-D-Maltoside (ODM), decyl beta-D-maltopyranoside (DMP), cyclohexyl-ethanoyl-maltoside, n-decyl- and n-dodecyl-sucrose, and mono- and di-fatty acid esters of PEG 300, 400, or 1750. Anionic surfactants can include but are not limited to sodium lauryl sulfate, fatty acid salts, sodium laureth sulfate, dioctyl sodium sulfosuccinate. Cationic surfactants can include but are not limited to Phosphatidylcholine (Lecithin), cetrimide, cetrimonium bromide, benethonium chloride, dimethyldioctadecyl ammonium chloride, tetradecyl trimethyl ammonium bromide, cetylpyridinium chloride, esterquat, and benzalkonium chloride. Zwitterionic surfactants can include but are not limited to Cocamidopropyl betaine, (3-[(3-Cholamidopropyl)dimethylammonio]-1-propanesulfonate) and cocamidopropyl hydroxysultaine, phosphatidylserine, phosphatidylethanolamine, phosphatidylcholine, and sphingomyelins.

Solvents that can be used include water-soluble organic solvents. Water-soluble organic solvents include but are not limited to polyethylene glycol 200, polyethylene glycol 300, polyethylene glycol 400, ethanol, propylene glycol, glycerin, N-methyl-2-pyrrolidone, dimethylacetamide, and dimethylsulfoxide.

Tonicity modifiers that can be used include but are not limited to dextrose, sucrose, mannitol, glycerin, sodium chloride, and potassium chloride.

pH modifiers that can be used include but are not limited to citric acid and its salts, salts of phosphoric acid, tartatic acid, lactic acid, glycolic acid, sodium hydroxide, phosphoric acid, sulfuric acid, oxalic acid and hydrochloric acid.

Anti-oxidants that can be used include but are not limited to ascorbic acid, butylated hydroxyanisole, Butylhydroxytoluene, Vitamin A, vitamin E, α-tocopherol, thioglycerol, cysteine, acetylcysteine, cystine, dithioerythreitol, dithiothreitol, glutathione, Sodium bisulfite, Sodium metabisulfite, thiourea, uric acid, melatonin, propyl gallate, tertiary butylhydroquinone and combinations thereof.

Emulsifiers that can be used include but are not limited to Glyceryl Monostearate, Isopropyl Palmitate, Polyethylene Glycol 400 Monostearate, as well as the compounds listed as surfactants and combinations thereof.

Preservatives that can be used include but are not limited to benzoic acid, sorbic acid, boric acid, methylparaben, ethylparaben, propylparaben, butylparaben, sodium benzoate, sodium propionate, phenyl ethyl alcohol, chlorobutanol, benzyl alcohol, potassium sorbate, phenol, chlorocresol, o-phenyl phenol, thiomersal, nitromersol, phenylmercuric nitrate, phenylmercuric acetate, benzalkonium and combinations thereof.

Excipients can include at least one solvent. Solvents used can include but are not limited to water, ethanol, dimethylsulfoxide, ethyl lactate, ethyl acetate, benzyl alcohol, benzyl benzoate, triacetin, N-methylpyrrolidone, 2-pyrrolidone, propylene carbonate, polyethylene glycol (PEG200), polyethylene glycol (PEG400), glycofurol and combinations thereof.

A medical device, which herein may refer to a medical device and/or a medical device pouch or envelope, can further comprise a biological agent. In an aspect, a disclosed aqueous lubricious composition can further comprise a biologically active agent. In an aspect, a medical device and/or a medical device pouch or envelope, the aqueous lubricious composition or a combination thereof can comprise more than one biologically active agents. Exemplary biologically active agents include, without limitation, small molecule drugs, peptides, proteins, growth factors, hormones, antibodies, agonists, antagonists, anti-bacterial and/or anti-fungal agents.

Biologically active agents that can be incorporated into or with disclosed compositions include but are not limited to, antiandrogens, antibacterial, antioestrogens, androgens and anabolic agents, antibiotics, antimigraine drugs, antihistamines, antianxiety drugs, antidiuretics, antihistamines, antirheumatoid agents, antigens, analgesics, antidepressants, antiinflammatories, anesthetics, aminoglycosides antibodies, antiviral, adrenergic stimulants, anticonvulsants, antiangina agents, antiarrhythmics, antimalarials, anti-mitotic, anthelmintics, anoretic agents, antitussives, antipruritics, antipyretics, anti-alzheimer's agents, anti-Parkinson's agents, antiemetics and antinauseants, antihypertensives, anticoagulants, antifungals, antimicrobials, allergens, antidiarrheals, antihyperuricaemia agents, adrenergic stimulants, antiparasitic agents, antiproliferative agents, antipsychotic drugs, antithyroid agents, beta-adrenergic blocking agents, bronchodilators; bronchospasm relaxants, blood clotting factors, blood coagulation factors, cytotoxic agents, cytostatic agents, chemotherapeutics, clot inhibitors, clot dissolving agents, cells, CNS stimulants, Corticosteroids, calcium channel blockers, cofactors, ceramides, cardiotonic glycosides, cytokines (e.g., lymphokines, monokines, chemokines); colony stimulating factors (e.g., GCSF, GM-CSF, MCSF); dermatological agents, decongestants, diuretics, expectorants, endectocide agents, growth factors, hemostatic agents, hypoglycemic agents, hormones and hormone analogs, hypercalcemia, Hypnotics, interleukins (IL-2, IL-3, IL-4, IL-6); interferons (.beta.-IFN, .alpha.-IFN and .gamma.-IFN); immunosuppressants, muscle relaxants, microorganisms, non-steroidal anti-inflammatory agents, nucleic acids, nutritional agents, neuromuscular blocking agents, neuroleptics, Neurotoxins, nutraceuticals, oligonucleotides, oestrogens, obstetric drugs, ovulation inducers, opioids, progestogens, pituitary hormones, Pituitary inhibitors proteins, peptides, polysaccharides, protease inhibitors, prostaglandins, quinolones, reductase inhibitors, sulfa drugs, sclerosant, sedatives, sodium channel blockers, steroids, steroidal anti-inflammatory agents, smoking cessation agents, toxins, thrombolytic agents, thyroid hormones, tumor necrosis factor; vesicles, vitamins, viruses, vasodilators, vaccines

Additional representative examples of biologically active agents that may be suitable for use in the compositions of the present invention include, but are not limited to: Antidiarrheals such as diphenoxylate, loperamide and hyoscyamine; Antihypertensives such as hydralazine, minoxidil, captopril, enalapril, clonidine, prazosin, debrisoquine, diazoxide, guanethidine, methyldopa, reserpine, trimethaphan; Calcium channel blockers such as diltiazem, felodipine, amlodipine, nitrendipine, nifedipine and verapamil; Antiarrhythmics such as amiodarone, flecainide, disopyramide, procainamide, mexiletene and quinidine, Antiangina agents such as glyceryl trinitrate, erythrityl tetranitrate, pentaerythritol tetranitrate, mannitol hexanitrate, perhexilene, isosorbide dinitrate and nicorandil; Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate; Cardiotonic glycosides such as digoxin and other cardiac glycosides and theophylline derivatives; Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine; Vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; Antiproliferative agents such as paclitaxel, estradiol, actinomycin D, sirolimus, tacrolimus, everolimus, 5-fluorouracil and dexamethasone; Antimigraine preparations such as ergotanmine, dihydroergotamine, methysergide, pizotifen and sumatriptan; Anticoagulants and thrombolytic agents such as warfarin, dicoumarol, low molecular weight heparins such as enoxaparin, streptokinase and its active derivatives; Hemostatic agents such as aprotinin, tranexamic acid and protamine; Analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine, dihydrocodeine; acetylsalicylic acid (aspirin), paracetamol, synthetic alpha2-adrenoreceptor agonist, dexmedetomidine hydrochloride, flunixin meglumine, meperidine, phenylbutazone and phenazone; Immunosuppressants, antiproliferatives and cytostatic agents such as rapamycin (sirolimus) and its analogs (everolimus and tacrolimus); Neurotoxins such as capsaicin, botulinum toxin (botox); Hypnotics and sedatives such as the barbiturates amylobarbitone, butobarbitone and pentobarbitone and other hypnotics and sedatives such as chloral hydrate, chlormethiazole, hydroxyzine and meprobamate; Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam; Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium; Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline; CNS stimulants such as caffeine and 3-(2-aminobutyl) indole; Antipruritics can include compounds such as synthetic Janus Kinase (JAK) inhibitors, NK-1 receptor antagonists, antibodies that neutralize interleukin-31 (IL-31). These can include oclacitinib maleate, Serlopitant, and Lokivetmab, Anti-alzheimer's agents such as tacrine; Anti-Parkinson's agents such as amantadine, benserazide, carbidopa, levodopa, benztropine, biperiden, benzhexol, procyclidine and dopamine-2 agonists such as S(−)-2-(N-propyl-N-2-thienylethylamino)-5-hydroxytetralin (N-0923), Anticonvulsants such as phenytoin, valproic acid, primidone, phenobarbitone, methylphenobarbitone and carbamazepine, ethosuximide, methsuximide, phensuximide, sulthiame and clonazepam, Antiemetics and antinauseants such as the phenothiazines prochloperazine, thiethylperazine, a neurokinin (NK1) receptor antagonist, maropitant citrateand 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride; Non-steroidal anti-inflammatory agents including their racemic mixtures or individual enantiomers where applicable, preferably which can be formulated in combination with dermal and/or mucosal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, 6-chloro-α-methyl-9H-carbazole-2-acetic acid (carprofen), flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate; Antirheumatoid agents such as penicillamine, aurothioglucose, sodium aurothiomalate, methotrexate and auranofin; Muscle relaxants such as baclofen, diazepam, cyclobenzaprine hydrochloride, dantrolene, methocarbamol, orphenadrine and quinine; Agents used in gout and hyperuricaemia such as allopurinol, colchicine, probenecid and sulphinpyrazone; Oestrogens such as estradiol, oestriol, estrone, ethinylestradiol, mestranol, stilbestrol, dienestrol, epiestriol, estropipate and zeranol; Progesterone and other progestagens such as allylestrenol, dydrgesterone, lynestrenol, norgestrel, norethyndrel, norethisterone, norethisterone acetate, gestodene, levonorgestrel, medroxyprogesterone and megestrol; Antiandrogens such as cyproterone acetate and danazol; Antioestrogens such as tamoxifen and epitiostanol and the aromatase inhibitors, exemestane and 4-hydroxy-androstenedione and its derivatives; Androgens and anabolic agents such as testosterone, methyltestosterone, clostebol acetate, drostanolone, furazabol, nandrolone oxandrolone, stanozolol, trenbolone acetate, dihydro-testosterone, 17-(.alpha.-methyl-19-noriestosterone and fluoxymesterone; 5-alpha reductase inhibitors such as finasteride, turosteride, LY-191704 and MK-306; Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone hydrocortisone 17-butyrate, hydrocortisone 21-acetate, methylprednisolone, prednisolone, prednisolone 21-phosphate, prednisone, triamcinolone, triamcinolone acetonide; Glycosylated proteins, proteoglycans, glycosaminoglycans such as chondroitin sulfate; chitin, acetyl-glucosamine, hyaluronic acid; Complex carbohydrates such as glucans; Further examples of steroidal anti-inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol; Pituitary hormones and their active derivatives or analogs such as corticotrophin, thyrotropin, follicle stimulating hormone (FSH), a Gonadotropin-releasing hormone (GnRH) analog, u deslorelin acetate, cetrorelix acetate, Gonadorelin acetate, clomiphene, Human chorionic gonadotropin (HCG), luteinizing hormone (LH) and gonadotrophin releasing hormone (GnRH); Hypoglycemic agents such as insulin, chlorpropamide, glibenclamide, gliclazide, glipizide, tolazamide, tolbutamide and metformin; Thyroid hormones such as calcitonin, thyroxine and liothyronine and antithyroid agents such as carbimazole and propylthiouracil; Other miscellaneous hormone agents such as octreotide; Pituitary inhibitors such as bromocriptine; Ovulation inducers such as clomiphene; Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene; Antidiuretics such as desmopressin, lypressin and vasopressin including their active derivatives or analogs; Obstetric drugs including agents acting on the uterus such as ergometrine, oxytocin and gemeprost; Prostaglandins such as alprostadil (PGE1), prostacyclin (PGI2), dinoprost (prostaglandin F2-alpha) and misoprostol; Antimicrobials including the cephalosporins such as cephalexin, cefoxytin and cephalothin; Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin; Tetracyclines such as minocycline, chlortetracycline, tetracycline, demeclocycline, doxycycline, methacycline and oxytetracycline and other tetracycline-type antibiotics; Amnioglycoides such as amikacin, amikin sulfate, gentamicin, kanamycin, neomycin, netilmicin and tobramycin; Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione; Quinolones such as nalidixic acid, cinoxacin, ciprofloxacin, enoxacin and norfloxacin; Sulphonamides such as phthalysulphthiazole, sulfadoxine, sulphadiazine, sulphamethizole and sulphamethoxazole; Sulphones such as dapsone; Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, secnidazole, ornidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds, silver sulfadiazine, silver, nanoparticulate silver, silver nitrate, silver zeolites, silver cations, AgPO3 Ag3PO₄, Ag4P207, Exsalt®SD7 (Exciton Technologies) Exsalt®T7 (Exciton Technologies); Lincomycin Hydrochloride, tricyclic tetrahydroquinoline antibacterial agents, 8-pyrazinyl-S-spiropyrimidinetrione-oxazinoquinoline derivatives, 3-spiropyrimidinetrione-quinoline derivatives, thiadiazol-spiropyrimidinetrione-quinoline derivatives, (2R,4S,4aS)-10-fluoro-2,4-dimethyl-8-(4-methyloxazol-2-yl)-2,4,4a,6-tetra-hydro-1H,1′H-spiro[[1,4]oxazino[4,3-a]quinoline-5,5′-pyrimidine]-2′,4′,6′(−3′H)-trione, (2R,4S,4aS)-9,10-difluoro-2,4-dimethyl-8-(3-methylisoxazol-5-yl)-2,4,4a,6-tetrahydro-1H, 1′H-spiro[[1,4]oxazino[4,3-a]quinoline-5,5′-pyrimidine]-2′,-4′,6′(3′H)-trione, (2R,4S,4aS)-10-fluoro-2,4-dimethyl-8-(oxazol-2-yl)-2,4,4a,6-tetrahydro-1H-,1′H-spiro[[1,4]oxazino[4,3-a]quinoline-5,5′-pyrimidine]-2′,4′,6′(3′H)-tri-one, (2R,4S,4aS)-9,10-difluoro-2,4-dimethyl-8-(2-methyloxazol-5-yl)-2,4,4a,6-t-etrahydro-1H,1′H-spiro[[1,4]oxazino[4,3-a]quinoline-5,5′-pyrimidine]-2′,4′-,6′(3′H)-trione, (2R,4S,4aS)-9,10-difluoro-2,4-dimethyl-8-(oxazol-4-yl)-2,4,4a,6-tetrahydr-o-1H,1′H-spiro[[1,4]oxazino[4,3-a]quinoline-5,5′-pyrimidine]-2′,4′,6′(3′H)-trione, (2R,4S,4aS)-9-fluoro-2,4-dimethyl-8-(4-methyloxazol-2-yl)-2,4,4a,6-tetrah-ydro-1H,1′H-spiro[[1,4]oxazino[4,3-a]quinoline-5,5′-pyrimidine]-2′,4′,6′(3′H)-trione, (2R,4S,4aS)-9,10-difluoro-8-(4-(4-fluorophenyl) oxazol-5-yl)-2,4-dimethyl-2,4,4a,6-tetrahydro-1H,1′H-spiro[[1,4]oxazino[4,3-a]quinoline-5,5′-pyrimid-ine]-2′,4′,6′(3′H)-trione, (2S,4R,4aR)-2,4-dimethyl-8-(oxazol-5-yl)-2,4,4a,6-tetrahydro-1H,1′H-spiro-[[1,4]oxazino[4,3-a]quinoline-5,5′-pyrimidine]-2′,4′,6′(3′H)-trione, (2S,4R,4aR)-8-(4-ethyloxazol-2-yl)-9,10-difluoro-2,4-dimethyl-2,4,4a,6-the-trahydro-1H,1′H-spiro[[1,4]oxazino[4,3-a]quinoline-5,5′-pyrimidine]-2′,4′,-6′(3′H)-trione, (2R,4S,4aS)-9,10-difluoro-2,4-dimethyl-8-(oxazol-2-yl)-2,4,4a,6-tetrahydr-o-1H,1′H-spiro[[1,4]oxazino[4,3-a]quinoline-5,5′-pyrimidine]-2′,4′,6′(3′H)-trione, benzoyl peroxide; Antituberculosis drugs such as ethambutol, isoniazid, pyrazinamide, rifampicin and clofazimine; Antimalarials such as primaquine, pyrimethamine, chloroquine, hydroxychloroquine, quinine, mefloquine and halofantrine; compounds such as Azithromycin, Aztreonam, Cefaclor, Cefadroxil, Cefazolin, Cefdinir, Cefepime Hydrochloride, (cefoperazone sodium, Ceftaroline fosamil, avibactam, Ceftazidime sodium, Ceftibuten, ceftiofur, Tazobactam, cefovecin sodium [(6R,7R)-7-[[(2Z)-(2-amino-4-thiazolyl)(methoxyimino) acetyl]amino]-8-oxo-3-[(2S)-tetrahydro-2-furanyl]-5-thia-1-azabicyclo[4.2.0]oct-2-ene-2-carboxylic acid, monosodium salt] Cefuroxime Axetil, Cefuroxime, Cephalexin, Chloramphenicol Sodium, Ciprofloxacin HCl, Clarithromycin, Clindamycin hydrochloride, Clindamycin Palmitate hydrochloride, Clindamycin phosphate, Dalbavancin Hydrochloride, Daptomycin, Demeclocycline hydrochloride, Dicloxacillin, Doripenem, Doxycycline, Doxycycline calcium, Doxycycline hyclate, Doxycycline monohydrate, Ertapenem sodium, Erythromycin, Erythromycin Ethylsuccinate, Erythromycin lactobionate, Erythromycin stearate, Erythromycin, Fosfomycin tromethamine, Gemifloxacin mesylate, Gentamicin Sulfate, Imipenem, Kanamycin, Levofloxacin, Lincomycin hydrochloride, Linezolid, Meropenem, Methenamine Hippurate, Metronidazole, Metronidazole, Micafungin sodium, Minocycline Hydrochloride, Minocycline, Moxifloxacin hydrochloride, Nafcillin, Nalidixic acid, Neomycin Sulfate, Nitrofurantoin, Norfloxacin, Ofloxacin, Oritavancin diphosphate, Oxacillin, Penicillin G, Penicillin G benzathine, Penicillin G Sodium, Penicillin V Potassium, Piperacillin Sodium, Polymyxin B Sulfate, Quinupristin, dalfopristin, Spectinomycin hydrochloride, Streptomycin, Sulfamethoxazole, Tedizolid Phosphate, Telavancin, Telithromycin, Tetracycline Hydrochloride, Sulfate, Tobramycin, Trimethoprim Ticarcillin disodium, Tigecycline, Tobramycin hydrochloride, tulathromycin, Vancomycin hydrochloride.

Antiviral agents may be included in disclosed compositions, where exemplary antiviral agents include but are not limited to acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. Other suitable biologically active agents include Anthelmintics such as mebendazole, thiabendazole, niclosamide, praziquantel, pyrantel embonate and diethylcarbamazine; Cytotoxic agents such as plicamycin, cyclophosphamide, dacarbazine, fluorouracil and its prodrugs (described, for example, in International Journal of Pharmaceutics, 111, 223-233 (1994)), methotrexate, procarbazine, 6-mercaptopurine and mucophenolic acid; Anorectic and weight reducing agents including dexfenflurarnine, fenfluramine, diethylpropion, mazindol and phentermine; Agents used in hypercalcaemia such as calcitriol, dihydrotachysterol and their active derivatives or analogs; Antitussives such as ethylmorphine, dextromethorphan and pholcodine; Antiparasitic and Endectocide agents such as moxidectin, Ivermectin, Niclosamide, Praziquantel, Pyrantel, Pyrvinium, Albendazole, Flubendazole, Mebendazole, Thiabendazole.

Compositions of the present disclosure may include but are not limited to: an expectorant such as carbolcysteine, bromihexine, emetine, quanifesin, ipecacuanha and saponins; decongestants such as phenylephrine, phenylpropanolamine and pseudoephedrine; Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives; Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine; Local anaesthetics such as benzocaine, bupivacaine, amethocaine, lignocaine, lidocaine, cocaine, cinchocaine, dibucaine, mepivacaine, prilocaine, etidocaine, veratridine (specific c-fiber blocker) and procaine; Stratum corneum lipids, such as ceramides, cholesterol and free fatty acids, for improved skin barrier repair [Man, et al. J. Invest. Dermatol., 106 (5), 1096, (1996)]; Neuromuscular blocking agents such as suxamethonium, alcuronium, pancuronium, atracurium, gallamine, tubocurarine and vecuronium; sclerocing agents or sclerosants may be a surfactant or it may be selected from the group consisting of ethanol, dimethyl sulfoxide, sucrose, sodium chloride, dextrose, glycerin, minocycline, tetracycline, doxycycline, polidocanol, sodium tetradecyl sulfate, sodium morrhuate, and sotradecol. an angiogenesis inhibitor; a 5-lipoxygenase inhibitor or antagonist; a chemokine receptor antagonist; a cell cycle inhibitor; a taxane; an anti-microtubule agent; paclitaxel; an analogue or derivative of paclitaxel; a vinca alkaloid; camptothecin or an analogue or derivative thereof; a podophyllotoxin, wherein the podophyllotoxin may be an etoposide or an analogue or derivative thereof; an anthracycline, wherein the anthracycline may be doxorubicin or an analogue or derivative thereof or the anthracycline may be mitoxantrone or an analogue or derivative thereof; a platinum compound; a nitrosourea; a nitroimidazole; a folic acid antagonist; a cytidine analogue; a pyrimidine analogue; a fluoropyrimidine analogue; a purine analogue; a nitrogen mustard or an analogue or derivative thereof; a hydroxyurea; a mytomicin or an analogue or derivative thereof; an alkyl sulfonate; a benzamide or an analogue or derivative thereof; a nicotinamide or an analogue or derivative thereof; a halogenated sugar or an analogue or derivative thereof; a DNA alkylating agent; an anti-microtubule agent; a topoisomerase inhibitor; a DNA cleaving agent; an antimetabolite; a nucleotide interconversion inhibitor; a hydroorotate dehydrogenase inhibitor; a DNA intercalation agent; an RNA synthesis inhibitor; a pyrimidine synthesis inhibitor; a cyclin dependent protein kinase inhibitor; an epidermal growth factor kinase inhibitor; an elastase inhibitor; a factor Xa inhibitor; a farnesyltransferase inhibitor; a fibrinogen antagonist; a guanylate cyclase stimulant; a heat shock protein 90 antagonist; which may be a geldanamycin or an analogue or derivative thereof; a guanylate cyclase stimulant; a HMGCoA reductase inhibitor, which may be simvastatin or an analogue or derivative thereof; an IKK2 inhibitor; an IL-1 antagonist; an ICE antagonist; an IRAK antagonist; an IL-4 agonist; an immunomodulatory agent; sirolimus or an analogue or derivative thereof; everolimus or an analogue or derivative thereof; tacrolimus or an analogue or derivative thereof; biolmus or an analogue or derivative thereof; tresperimus or an analogue or derivative thereof; auranofin or an analogue or derivative thereof; 27-0-demethylrapamycin or an analogue or derivative thereof; gusperimus or an analogue or derivative thereof; pimecrolimus or an analogue or derivative thereof; ABT-578 or an analogue or derivative thereof; an inosine monophosphate dehydrogenase (IMPDH) inhibitor, which may be mycophenolic acid or an analogue or derivative thereof or 1-.alpha.-25 dihydroxy vitamin D.sub.3 or an analogue or derivative thereof; a leukotriene inhibitor; an MCP-1 antagonist; an MMP inhibitor; an NF kappa B inhibitor, which may be Bay 11-7082; an NO antagonist; a p38 MAP kinase inhibitor, which may be SB 202190; a phosphodiesterase inhibitor; a TGF-.beta. inhibitor; a thromboxane A2 antagonist; a TNF-.alpha. antagonist; a TACE inhibitor; a tyrosine kinase inhibitor; vitronectin inhibitor; a fibroblast growth factor inhibitor; a protein kinase inhibitor; a PDGF receptor kinase inhibitor; an endothelial growth factor receptor kinase inhibitor; a retinoic acid receptor antagonist; a platelet derived growth factor receptor kinase inhibitor; a fibrinogen antagonist; an antimycotic agent; sulconizole; a bisphosphonate; a phospholipase A1 inhibitor; a histamine H1/H2/H3 receptor antagonist; a macrolide antibiotic; a GPIIb/IIIa receptor antagonist; an endothelin receptor antagonist; a peroxisome proliferator-activated receptor agonist; an estrogen receptor agent; a somastostatin analogue; a neurokinin 1 antagonist; a neurokinin 3 antagonist; a VLA-4 antagonist;

an osteoclast inhibitor; a DNA topoisomerase ATP hydrolyzing inhibitor; an angiotensin I converting enzyme inhibitor; an angiotensin II antagonist; an enkephalinase inhibitor; a peroxisome proliferator-activated receptor gamma agonist insulin sensitizer; a protein kinase C inhibitor; a ROCK (rho-associated kinase) inhibitor; a CXCR3 inhibitor; Itk inhibitor; a cytosolic phospholipase A.sub.2-.alpha. inhibitor; a PPAR agonist; an immunosuppressant; an Erb inhibitor; an apoptosis agonist; a lipocortin agonist; a VCAM-1 antagonist; a collagen antagonist; an .alpha.-2 integrin antagonist; a TNF-.alpha. inhibitor; a nitric oxide inhibitor; and a cathepsin inhibitor. anti-fibrin and fibrinolytic agents, including plasmin, streptokinase, single chain urokinase, urokinase, t-PA (tissue type plasminogen activator), aminocaproic acid; anti-platelet agents including, aspirin, prostacyclins (and analogues); glycoprotein IIb/IIIa agents including monoclonal antibodies, peptides (e.g. ReoPro, Cilastagel, eptifibatide, tirofiban, ticlopidine, Vapiprost, dipyridamole, forskolin, angiopeptin, argatroban), thromboxane inhibitors; anti-thrombin and anti-coagulant agents, including dextan, heparin, LMW heparin (Enoxaparin, Dalteparin), hirudin, recombinant hirudin, anti-thrombin, synthetic antithrombins, thrombin inhibitors, Warfarin (and other coumarins); anti-mitotic, antiproliferative and cytostatic agents, including vincristine, vinblastine, paclitaxel, methotrexate, cisplatin, fluorouracil, rapamycin, azathioprine, cyclophosphamide, mycophenolic acid, corticosteroids, colchicine, nitroprusside; antiangiogenic and angiostatic agents, including paclitaxel, angiostatin and endostatin; genetic materials and oligonucleotides; ACE inhibitors (e.g. Cilazapril, Lisinopril, Captopril); growth factor (e.g. VEGF, FGF) antagonists; antioxidants and vitamins (e.g. Probucol, Tocopherol); calcium channel blockers (e.g. nifedipine); fish oil (omega 3-fatty acid); phosphodiesterase inhibitors (e.g. dipyridamole); nitric acid donor (e.g. Molsidomine); somatostatin analogues (e.g. angiopeptin); immunosuppressives and anti-inflammatory agents (e.g. prednisolone, glucocorticoid and dexamethasone); antimicrobials (e.g. rifamycin) and radionuclides, including alpha, beta and gamma emitting isotopes (e.g. Re-188, Re-186, I-125, Y-90); COX-2 inhibitors such as Celecoxib and Vioxx; kinase inhibitors, such as epidermal growth factor kinase inhibitor, tyrosine kinase inhibitors, MAP kinase inhibitors protein transferase inhibitors, Resten-NG, Smoking cessation agents such as nicotine, bupropion and ibogaine; Insecticides and other pesticides which are suitable for local application; Dermatological agents, such as vitamins A, C, B1, B2, B6, B 12, B 12.alpha . . . , and E, vitamin E acetate and vitamin E sorbate; Allergens for desensitization such as house, dust or mite allergens; Nutritional agents and neutraceuticals, such as vitamins, essential amino acids and fats; Macromolecular pharmacologically active agents such as proteins, enzymes, peptides, polysaccharides (such as cellulose, amylose, dextran, chitin), nucleic acids, cells, tissues, and the like; Bone mending biochemicals such as calcium carbonate, calcium phosphate, tricalcium phosphate, hydroxyapetite or bone morphogenic protein (BMP); Angiogenic growth factors such as Vascular Endothelial Growth Factor (VEGF) and epidermal growth factor (EFG), cytokines interleukins, fibroblasts and cytotaxic chemicals; and Keratolytics such as the alpha-hydroxy acids, glycolic acid and salicylic acid; and DNA, RNA or other oligonucleotides. Vaccines that contain Hendra virus (HeV) G glycoprotein and/or Nipah virus G glycoprotein, Lutenising Hormone Releasing Hormone (LHRH) peptide, LHRH-diphtheria toxoid conjugate, porcine circovirus type 2 (PCV2) antigen, a porcine reproductive and respiratory syndrome virus antigen, Mycoplasma hyopneumoniae protein antigen. Proteins or protein fragments, for example ORFI Torque teno virus protein, or other TTV proteins or fragments, antigens against Aeromonas salmonicida, antigens against Vibrio anguillarum, and antigens against V. salmonicida. Growth factors include but are not limited to Vascular Endothelial Growth Factor (VEGF) and epidermal growth factor (EFG), Growth Differentiation Factors (GDFs), Fibroblast Growth Factors (FGF-1 through FGF-23), Osteoprotegerin, Cartilage Derived Morphogenic Proteins (CDMPs, which can be a foundation for soft or hard tissue), Lim Mineralization Proteins (LMPs) Interleukins (IL-1 through IL-13), Insulin-like Growth Factor-1, Connective Tissue Growth Factor (CTGF), platelet derived growth factor (PDGF), nerve growth factors, neutrophins Brain-derived neurotrophic factor (BDNF), Nerve growth factor (NGF), Neurotrophin-3 (NT-3), Neurotrophin-4 (NT-4)], Transforming growth factors (TGF-α, TGF-β), Tumor necrosis factor (TNF). Growth factor Agonists or antagonists as well as antibodies against these growth factors. Biologically active agents that can be used to treat macular degeneration include but are not limited to bevacizumab and ranibizumab. Biologically active agents can include ansamycin antibiotic. Ansamycin antibiotics can include but are not limited to rifampicin, geldanamycin, rifabutin, rifaximin, and rifamycin S.

In an aspect, an active agent is a protein, where that term includes peptides and polypeptides, sugar-modified protein such as glycoprotein, as well as functional descriptions of protein classes such as antigen, enzyme, immunoglobulin and antibody. A disclosed composition may include but is not limited to a special delivery vehicle for the active agent, such as a virus or modified virus, where the active agent, such as a protein or polynucleotide, is contained within or expressed by the special delivery vehicle.

In an aspect, an active agent, also referred to herein as a biologically active agent, is a PD-L1 inhibitor. A PD-L1 inhibitor can include but is not limited to Atezolizumab, Avelumab, Durvalumab, LY3300054 (Eli Lilly and Company), and monoclonal antibodies or monoclonal antibody conjugates that act as a PD-L1 inhibitor

In an aspect, an active agent is a PD-1 inhibitor A PD-1 inhibitor can include but is not limited to pembrolizumab, Nivolumab, Cemiplimab and monoclonal antibodies or monoclonal antibody conjugates that act as a PD-1 inhibitors.

In an aspect, an active agent is a CTLA-4 inhibitor. A CTLA-4 inhibitor can include but is not limited to Ipilimumab, AGEN1884 and monoclonal antibodies or monoclonal antibody conjugates that act as a CTLA-4 inhibitor.

In an aspect, an active agent is a compound that is used to treat non-muscle invasive bladder cancer. The compounds that can be used include but are not limited to non-live immunologically active Bacillus Calmette-Guerin (BCG) subcomponents that include BCG cell wall and various BCG proteins and antigens, an IL-2 fusion protein such as ALT-801 (Altor Bioscience), Oportuzumab monatox (Sesen Bio), sunitinib (Pfizer), enzalutamide, ethacrynic acid, Imiquimod and tamoxifen, ALT-803 (Altor Bioscience), and Lenalidomide.

In an aspect, an active agent is an antibody drug conjugate. The antibody drug conjugate can include but is not limited to trastuzumab emtansine, Sacituzumab govitecan, Enfortumab vedotin, ASG-15ME, Gemtuzumab ozogamicin, Brentuximab vedotin, Trastuzumab emtansine, and Inotuzumab ozogamicin.

In an aspect, an active agent is a small molecule protein kinase inhibitor. The small molecule protein kinase inhibitor can include but is not limited to abenaciclib, acalabrutini, afatinib, alectinib, axitinib, baricitinib, binimetinib, bosutinib, brigatinib, cabozantinib, ceritinib, cobimetinib, crisotinib, dabrafenib, dacomitinib, dasatinib, encorafenib, erdafitinib, erlotinib, everolimus, fostamatinib, gefitinib, gilteritinib, ibrutinib, imatinib, lapatinib, larotrectinib, lenvatinib, lorlatinib, midostaurin, neratinib, netarsudil, nilotinib, nintedanib, osimertinib, palbociclib, Pazopanib, Ponatinib, Regorafenib, Ribociclib, Ruxolitinib, Sirolimus, Sorafenib, Sunitinib, Temsirolimus, Tofacitinib, Trametinib, Vandetanib, and Vemurafenib.

One or more disclosed aqueous lubricious compositions may be used to coat a medical device. In one aspect, a method of applying an aqueous lubricious composition to a medical device involves pouring a disclosed aqueous lubricious composition over the device. In an aspect, a method of applying a disclosed aqueous lubricious composition to a medical device involves using a syringe to withdraw a disclosed aqueous lubricious composition from its container and then expelling the aqueous lubricious composition from the syringe onto one or more surfaces of a medical device. In an aspect, a medical device can be dipped into a disclosed aqueous lubricious composition. In an aspect, a medical device can be immersed into a disclosed aqueous lubricious composition. In an aspect, the entire medical device can be coated. In another aspect a portion of the medical device can be coated. In an aspect, one or more portions of, or all of the, surfaces of a medical can be coated with an aqueous lubricious composition disclosed herein.

Following application of a disclosed aqueous lubricious composition to a medical device, the medical device comprising the aqueous lubricious composition is inserted into the desired location in the body. In one aspect, the desired location in the body is through a body orifice. In another aspect, the desired location in the body is through a surgical incision. In another aspect, the desired location in the body is through a traumatic injury. In one aspect, excess aqueous lubricious composition is removed from the medical device comprising the aqueous lubricious composition after which the medical device comprising the aqueous lubricious composition is inserted into the desired location in the body.

A kit of the present disclosure comprises a medical device and an aqueous lubricious solution. In an aspect, a kit comprises a medical device, an aqueous lubricious composition and a container, which may contain the aqueous lubricious composition, or a container into which the aqueous lubricious composition can be poured. In an aspect, a kit comprises a medical device and an aqueous lubricious composition and a syringe. In another aspect, a kit comprises a medical device and an aqueous lubricious solution, a container and a syringe. In another aspect, a kit comprises an aqueous lubricious composition and a container. In another aspect, the kit comprises an aqueous lubricious composition, a container and a syringe. In an aspect, the aqueous lubricious composition is contained by a syringe. In an aspect, a aqueous lubricious composition is provided in the container.

There are numerous methods by which the kits, the aqueous lubricious solutions and medical devices described in the present disclosure can be used in addition to the methods described above. In one aspect, medical device can be removed from its sterile primary packaging, the container which comprises the sterile aqueous lubricious composition can be opened, the sterile aqueous lubricious composition can be applied to the medical device and the medical device can be inserted into the desired location in the body. In another aspect, the container which comprises the sterile aqueous lubricious composition can be opened, the medical device can be removed from its sterile primary packaging, the sterile aqueous lubricious composition can be applied to the medical device and the medical device can be inserted into the desired location in the body.

In an aspect, a medical device can be removed from its sterile primary packaging, a container which comprises the sterile aqueous lubricious composition can be opened, the medical device can be immersed into the sterile aqueous lubricious composition for a period of time, after which the medical device is removed from the sterile aqueous lubricious composition and the medical device can be inserted into the desired location in the body. In an aspect, the container which comprises the sterile aqueous lubricious composition can be opened, the medical device can be removed from its sterile primary packaging, the medical device can be immersed into the sterile aqueous lubricious composition for a period of time, after which the medical device is removed from the sterile aqueous lubricious composition and the medical device can be inserted into the desired location in the body. In an aspect, the medical device can be immersed in the sterile aqueous lubricious composition for less than about 15 seconds. In an aspect, the medical device can be immersed in the sterile aqueous lubricious composition for longer than about 15 seconds. In an aspect, a medical device can be immersed in a disclosed sterile aqueous lubricious composition for less than about 60 seconds. In an aspect, a medical device can be immersed in a disclosed sterile aqueous lubricious composition for greater than about 60 seconds. In an aspect, a medical device can be immersed in a disclosed sterile aqueous lubricious composition for greater than about 5 seconds to about 5 minutes. In an aspect, a medical device can be immersed in a disclosed sterile aqueous lubricious composition for greater than about 10 seconds to about 4 minutes. In an aspect, a coated medical device can be moved in such a manner to remove some of the applied sterile aqueous lubricious composition prior to inserting it into the desired location in the body. In an aspect, one medical device can be coated after which it is combined with a second medical device. In an aspect, one medical device can be coated, the second medical device can be coated and then the two medical devices can be combined. In an aspect, the medical devices can be the same. In another aspect, the medical devices can be different. In an aspect, a method of use may comprise flushing the location where the medical device is to be inserted or the site where the medical device has been inserted and comprises the coated medical device, with saline or a solution that comprises an antibiotic or a combination of these flushing methods. In an aspect, a method may comprise using sutures or surgical staples to close the location in the body into which the coated medical device was placed or inserted.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

An aqueous lubricious composition was prepared by adding sodium hyaluronate (Shiseido D30475, Mw about 800K) at about a 1.5% (w/v) concentration into deionized water and mixing the composition on a roller mill until homogenous. A multifilament warp knit mesh (approximately 100 mm×100 mm) composed of a glycolide copolymer (Glycoprene® 935, Poly-Med, Inc., USA) was immersed into the sodium hyaluronate solution. The coated mesh was withdrawn from the solution after about 15 seconds and held above the sodium hyaluronate solution to allow the excess coating solution to drip off.

### Example 2

An octanethiol derivatized hyaluronic acid polymer was prepared according to U.S. Pat. No. 11,440,976. An aqueous lubricious composition was prepared by adding the octanethiol derivatized hyaluronic acid polymer (approximately 70% substitution) at about a 1.0% (w/v) concentration into deionized water and mixing the composition on a roller mill until homogenous. A multifilament warp knit mesh (approximately 100 mm×100 mm) composed of a glycolide copolymer (Glycoprene® 935 Poly-Med, Inc., USA) was immersed into the derivatized hyaluronate solution. The coated mesh was withdrawn from the solution after about 15 seconds.

### Example 3

A mercaprobenzoic acid derivatized hyaluronic acid polymer was prepared according to U.S. Pat. No. 11,440,976. An aqueous lubricious composition was prepared by adding the mercaptobenzoic acid derivatized hyaluronic acid polymer (approximately 80% substitution) at about a 1.0% (w/v) concentration into deionized water and mixing the composition on a roller mill until homogenous. A multifilament warp knit mesh (approximately 100 mm×100 mm) composed of a glycolide copolymer (Glycoprene® 935) was immersed into the derivatized hyaluronate solution. The coated mesh was withdrawn from the solution after about 15 seconds.

### Example 4

An aqueous lubricious composition is prepared by adding sodium hyaluronate (Shiseido D30475, Mw about 800K) at about a 1.5% (w/v) concentration into deionized water and mixing the composition on a roller mill until homogenous. The aqueous lubricious composition is autoclaved to provide a sterile solution. A sterile implantable cardioverter defibrillator is inserted into a sterile pouch made from a knitted glycolide containing copolymer. The implantable cardioverter defibrillator/pouch is immersed into the lubricious solution. After 15 seconds, the coated device combination is removed from the coating and the initial excess solution is allowed to drip from the device. The coated device is then inserted into a surgically created pocket.

### Example 5

An aqueous lubricious composition is prepared by adding mercaprobenzoic acid derivatized hyaluronic acid polymer at about a 1.0% (w/v) concentration into deionized water and mixing the composition on a roller mill until homogenous. The aqueous lubricious composition is autoclaved to provide a sterile solution. A sterile implantable cardioverter defibrillator is inserted into a sterile pouch made from a knitted glycolide containing copolymer. The implantable cardioverter defibrillator/pouch is immersed into the lubricious solution. After 15 seconds, the coated device combination is removed from the coating solution and the initial excess solution is allowed to drip from the device. The coated device is then inserted into a surgically created pocket.

### Example 6

An aqueous lubricious composition is prepared by adding sodium hyaluronate (Shiseido D30475, Mw about 800K) at about a 1.5% (w/v) concentration into deionized water and mixing the composition on a roller mill until homogenous. The aqueous lubricious composition is autoclaved to provide a sterile solution. A sterile pouch made from a knitted glycolide containing copolymer that further comprises minocycline and rifampin is immersed into the lubricious solution. After 15 seconds, the coated device combination is removed from the coating solution and the initial excess solution is allowed to drip from the device. The implantable cardioverter defibrillator is then inserted into the coated pouch. The coated device combination is then inserted into a surgically created pocket.

### Example 7

An aqueous lubricious composition is prepared by adding sodium hyaluronate (Shiseido D30475, Mw about 800K) at about a 1.5% (w/v) concentration into deionized water and mixing the composition on a roller mill until homogenous. The aqueous lubricious composition is autoclaved to provide a sterile solution. A sterile pouch made from a knitted glycolide containing copolymer that further comprises minocycline and rifampin is immersed into the lubricious solution. After 15 seconds, the coated device combination is removed from the coating solution and the initial excess solution is allowed to drip from the device. The implantable cardioverter defibrillator is then inserted into the coated pouch. The coated device combination is then inserted into a surgically created pocket. The surgical pocket is irrigated with saline.

### Example 8

An aqueous lubricious composition is prepared by adding sodium hyaluronate (Shiseido D30475, Mw about 800K) at about a 1.5% (w/v) concentration into deionized water and mixing the composition on a roller mill until homogenous. The aqueous lubricious composition is autoclaved to provide a sterile solution. A sterile warp knitted mesh comprising polydioxanone is immersed into the lubricious solution. After 15 seconds, the coated device is removed from the coating solution and the initial excess solution is allowed to drip from the device. The coated mesh is then placed into the surgical site.

All references disclosed herein, including patent references and non-patent references, are hereby incorporated by reference in their entirety as if each was incorporated individually.

It is to be understood that the terminology used herein is for the purpose of describing specific aspects only and is not intended to be limiting. It is further to be understood that unless specifically defined herein, the terminology used herein is to be given its traditional meaning as known in the relevant art.

Reference throughout this specification to “one aspect” or “an aspect” and variations thereof means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an aspect that includes all of the associated items or ideas and one or more other alternative aspects that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed disclosure.

Any headings used within this document are only being utilized to expedite its review by the reader, and should not be construed as limiting the disclosure or claims in any manner. Thus, the headings and Abstract of the Disclosure provided herein are for convenience only and do not interpret the scope or meaning of the aspects.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described disclosure. The publications discussed above and throughout the text are provided solely for their disclosure prior to the filing date of the present application. Nothing herein is to be construed as an admission that the inventors are not entitled to antedate any referenced publication by virtue of prior disclosure.

All patents, publications, scientific articles, web sites, and other documents and materials referenced or mentioned herein are indicative of the levels of skill of those skilled in the art to which the disclosure pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific aspects disclosed in the specification and the claims, but should be construed to include all possible aspects along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting aspects are within the following claims. The patent may not be interpreted to be limited to the specific examples or nonlimiting aspects or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

What is claimed is:

1) A kit comprising a sterile aqueous lubricant and a sterile medical device, wherein the sterile aqueous lubricant comprises a biocompatible, degradable polymer comprising hyaluronic acid, sodium hyaluronate, octanethiol derivatized hyaluronic acid, mercaprobenzoic acid derivatized hyaluronic acid, a hyaluronic acid derivative, or a hyaluronate derivative thereof.

2) The kit of claim 1, further comprising a sterile container into which the sterile aqueous lubricant can be dispensed.

3) The kit of claim 1 or 2, wherein the sterile medical device comprises a biologically active agent.

4) The kit of claim 3, wherein the biologically active agent comprises an antibiotic, an antiviral, a polypeptide, a polynucleotide, a hormone, an antibody-drug conjugate, an anti-cancer agent, a small molecule inhibitor, or any therapeutic agent thereof.

5) The kit of any one of claims 1-4, wherein the sterile medical device comprises a mesh, a catheter, a pouch, a cardiac implantable electronic device, a needle, a syringe, a stent, a suture, a replacement tissue, or a composition or device thereof.

6) The kit of any one of claims 1-5, wherein the sterile medical device further comprises a degradable copolymer selected from one or more monomers consisting of lactide, glycolide, ε-caprolactone, trimethylene carbonate, morpholine-dione, p-dioxanone and 1,5-dioxapan-2-one.

7) A composition comprising the sterile aqueous lubricant of any one of claims 1-6.

8) The composition of claim 7, further comprising a sterile carrier selected from water, saline, buffer, solubilizer, stabilizer, or a combination thereof.

9) A method of placing a coated sterile medical device into a subject, the method comprising:
a) applying a sterile aqueous lubricant to a sterile medical device, wherein the sterile aqueous lubricant comprises a biocompatible, degradable polymer and a sterile carrier, and wherein the polymer comprises hyaluronic acid, sodium hyaluronate, octanethiol derivatized hyaluronic acid, mercaprobenzoic acid derivatized hyaluronic acid, a hyaluronic acid derivative, or a hyaluronate derivative thereof;
b) inserting the coated sterile medical device into a surgically created site in the subject; and
c) closing the surgically created site that now contains the coated sterile medical device.

10) A method of placing a coated sterile medical device into a subject, the method comprising:
a. applying a sterile aqueous lubricant to a sterile medical device, wherein the sterile aqueous lubricant comprises a biocompatible, degradable polymer and a sterile carrier, and wherein the polymer comprises hyaluronic acid, sodium hyaluronate, octanethiol derivatized hyaluronic acid, mercaprobenzoic acid derivatized hyaluronic acid, a hyaluronic acid derivative, or a hyaluronate derivative thereof;
a) inserting the coated sterile medical device into a surgically created site in the subject;
b) irrigating the surgically created site that contains the coated medical device with saline; and
c) closing the surgically created site that now contains the coated sterile medical device.

11) The method of claim 9 or 10, wherein the sterile carrier is selected from water, saline, buffer, solubilizer, stabilizer, or a combination thereof.

12) The method of any one of claims 9-11, further comprising a sterile container into which the sterile aqueous lubricious composition can be dispensed.

13) The method of claim 9-12, wherein the coated sterile medical device comprises a biologically active agent.

14) The method of claim 13, wherein the biologically active agent comprises an antibiotic, an antiviral, a polypeptide, a polynucleotide, a hormone, an antibody-drug conjugate, an anti-cancer agent, a small molecule inhibitor, or any therapeutic agent thereof.

15) The method of any one of claims 9-14, wherein the sterile medical device comprises a mesh, a catheter, a pouch, a cardiac implantable electronic device, a needle, a syringe, a stent, a suture, a replacement tissue, or a composition or device thereof.

16) The method of any one of claims 9-15, wherein the sterile medical device further comprises a degradable copolymer selected from one or more monomers consisting of lactide, glycolide, ε-caprolactone, trimethylene carbonate, morpholine-dione, p-dioxanone and 1,5-dioxapan-2-one.
